(19) United States
(12) Patent Application Publication
(10) Pub. No.: US 2026/0232327 A1
(43) Pub. Date: Aug. 13, 2026

(54) METHOD OF SHAPING A SURFACE OF A BONE

(71) Applicant: TriMed, Inc., Santa Clarita, CA (US)

(72) Inventor: Robert J. Medoff, Kailua, HI (US)

(21) Appl. No.: 19/048,385

(22) Filed: Feb. 7, 2025

(51) Int. Cl. A61B 17/16; A61B 17/17; A61B 17/56

(52) U.S. Cl. CPC A61B 17/1659; A61B 17/1628; A61B 17/17; A61B 2017/564

(57) ABSTRACT

A method of shaping a surface of a bone including the steps of: a) obtaining a shaping tool with a turning axis and axially spaced proximal and distal ends and having: at least first and second discrete arms projecting away from the turning axis with at least a first circumferential gap defined between circumferentially adjacent of the at least first and second arms, the at least first circumferential gap extending axially towards the proximal end of the shaping tool to beyond the circumferentially adjacent of the at least first and second arms; and a cutting edge on each of the at least first and second arms moving in a cutting path as the shaping tool is advanced around the turning axis; and b) bearing the at least first and second cutting edges against the bone while advancing the shaping tool around the turning axis and thereby causing: i) the at least first and second cutting edges to remove bone material and produce a flat bone surface shape; and ii) removed bone material to move axially away from the flat bone surface shape and into the first circumferential gap.

### BACKGROUND OF THE INVENTION

### Field of the Invention

This invention relates to bone reconfiguration performed as during a surgical procedure and, more particularly, to a method of shaping an exposed region of a bone to produce a flat bone surface shape thereat.

### Background Art

Many different surgical procedures are performed that require reconfiguration of an exposed region of a bone, commonly carried out by using a driven rotary tool. The tool may take many different forms to strategically produce different surface contours, as to accommodate an implant, facilitate fusion, etc. In one category of these procedures, a bone region is “ground” to produce a flat bone surface shape.

There are cases in which a surgeon wants to create apposing flat surfaces. This is commonly done by cutting a bone freehand. However, it is difficult to define surfaces residing in planes that appose each other precisely at a desired angle.

Without limitation, joint fusion, such as at exemplary interphalangeal, metacarpalphalangeal, or metatarsalphalangeal joints, is carried out with initial bone reconfiguration to produce two flat surfaces that are placed in apposed relationship preparatory to fusion.

For example, as shown in FIGS. 1 and 2, fusion may be effected between a proximal phalanx/first bone 10 and a more distal phalanx/second bone 12 by reconfiguring cooperating surfaces 14, 16, respectively on the first and second bones, which cooperate at a joint 17, as shown in FIG. 1. More specifically, the surfaces 14, 16 are ground to respectively define flat configurations, as shown in FIG. 2, which, as shown in that Figure, are placed in apposed relationship and thereafter appropriately fixed through any of a number of different techniques and using different fusing components.

Typically, the reconfiguration of the bone surfaces 14, 16 involves exposing the surfaces 14, 16 adequately that they can be ground, as by using an exemplary, conventional cutting tool, shown at 18 in FIGS. 3-5. The tool 18 is a representative structure having limitations which are generally addressed by the present invention, as explained below.

The tool 18 has an elongate driving body/shank 20 with one end having a connector at 22 that is engageable with a rotary driver, shown schematically at 24 in FIG. 3. With the rotary driver 24 engaged with the tool 18 and operated, the tool 18 is driven around a lengthwise axis 26.

At the distal end of the tool 18 is a cutting head 28 having a disk-shaped body 30 with a radial array of teeth 32 emanating from adjacent the axis 26 and regularly circumferentially spaced therearound. The teeth 32 project axially from the distal region of the body 30 and each has a sharp, elongate cutting edge 34 at its free axial end. The cutting edges 34 are borne against a bone and separate discrete particles of bone as the cutting head 28 is advanced in the direction of the arrow 36 around the axis 26.

The cutting edges 34 reside in a common plane so as to produce a flat bone surface shape when borne against a bone and rotated.

To facilitate controlled grinding, the cutting tool 18 is provided with an axially extending guide passage 38 through the cutting head 28 and at least a portion of the body/shank 20.

As shown in FIG. 6, an elongate guide component 40 may be directed into a bone so as to be anchored therewithin. The cutting tool can then be directed over the elongate guide component 40 and guidingly turned, whereby the elongate guide component 40 dictates the location and orientation of the flat bone surface shape that is produced.

In the case of the bones 10, 12, depicted in FIGS. 1 and 2, the elongate guide component 40 can be directed in separate steps through the ends of the first and second bones 10, 12, and lengthwise therewithin, with alignment selected precisely to produce the flat bone surface shapes in FIG. 2. With the cutting tool 18 guided by the elongate guide component 40, the cutting tool can be operated through the rotary driver 24 to grind the bone ends 10, 12 to reshape the bone surfaces 14, 16 from the shape shown in FIG. 1, to that shown in FIG. 2.

Alternatively, the grinding of the surfaces 14, 16 can be carried out “freehand” without the assistance of an elongate guide component 40.

Again, it should be emphasized that there are numerous procedures requiring the localized formation of a flat bone surface shape. The depicted bones in FIGS. 1 and 2 and the fusion procedure depicted are exemplary in nature only.

Cutting tools having the same basic configuration as the cutting tool 18 have been commonly used by surgeons worldwide. However, this design has a number of inherent limitations.

Most significantly, the volume 42 between adjacent teeth 32 tends to capture bone particles that are removed during a grinding operation. While some of the particles may be centrifugally propelled away from the cutting head 28, there is generally a progressive accumulation of the particles in the volumes 42. Eventually, the bone particles may accumulate to the point that the volumes 42 are substantially full, whereby some, or all, of the cutting edges 34 are inhibited from cutting to any appreciable depth, or any depth at all.

This condition necessitates that the surgeon stop the procedure and manually clear the accumulated particles on the cutting head 28, after which the procedure can be continued. If the particles are compacted in the volumes 42, it may take a significant amount of time to clean the cutting head. Alternatively, the cutting tool 18 with the bone particle buildup may be separated from the rotary driver 24 and replaced with a clean tool.

The need to interrupt a surgical procedure and either clean the cutting head 28 or substitute another cutting tool 18 is an inconvenience that contributes to fatigue and also undesirably lengthens the time required to perform a surgical procedure.

Additionally, as the bone particles are accumulating in the volumes 42, the depth of cutting/grinding is progressively diminished. Accordingly, the surgeon may have to grind for an extended period.

As the cutting head 28 turns and removes bone particles, it also progressively heats the bone region against which it bears through frictional forces between the bone region and the teeth 32 and accumulated and compacted bone particles in the volumes 42. Thus, the longer the grinding tool 18 is operated, the greater the heat buildup. Heat buildup has the potential to damage bone and thus it is desirable to minimize frictional heat generation by carrying out efficient cutting in the shortest possible timeframe. This objective is frustrated by the progressive building up of the bone particles on the cutting head 28, which impairs its performance.

### SUMMARY OF THE INVENTION

In one form, the invention is directed to a method of shaping a surface of a bone. The method includes the steps of: obtaining a first shaping tool having a turning axis, and axially spaced proximal and distal ends; and bearing the at least first and second cutting edges against the bone while advancing the first shaping tool around the turning axis and thereby causing: a) the at least first and second cutting edges to remove bone material and produce a flat bone surface shape; and b) removed bone material to move axially away from the flat bone surface shape and into the first circumferential gap. The first shaping tool includes at least first and second discrete arms projecting away from the turning axis with at least a first circumferential gap defined between circumferentially adjacent of the at least first and second arms. The at least first circumferential gap extends axially towards the proximal end of the first shaping tool to beyond the circumferentially adjacent of the at least first and second arms. The at least first and second arms each has a cutting edge. The cutting edge on each of the at least first and second arms moves in a cutting path as the first shaping tool is advanced around the turning axis;

In one form, the cutting edges on the first and second arms have lengths projecting away from the turning axis that reside substantially in a single plane.

In one form, the at least first and second arms include at least a third arm.

In one form, the at least first and second arms include at least a third arm and a fourth arm.

In one form, the lengths of the cutting edges on the first and second arms are substantially straight and respectively extend along first and second lines.

In one form, the first and second lines intersect the turning axis for the first shaping tool.

In one form, the at least first and second arms are part of a cutting head and the first circumferential gap is axially unobstructed whereby removed bone material moves axially away from the flat bone surface shape and into and fully through the first circumferential gap and fully through the cutting head.

In one form, the first circumferential gap is defined between the first and second arms. The distal end of the first shaping tool is a leading end. The first arm has a leading end and a trailing end. The first arm has a radial distal end. A radially projecting dimension of the first arm to the radial distal end of the first arm is reduced between the leading end of the first arm and the trailing end of the first arm.

In one form, the first arm has circumferentially oppositely facing leading and trailing surfaces. The radially projecting dimension of the first arm between the oppositely facing leading and trailing surfaces changes between the leading end and trailing end of the first arm.

In one form, the leading surface of the first arm is substantially flat.

In one form, the leading surface of the first arm resides in a plane that is substantially parallel to the turning axis.

In one form, the plane is substantially coincident with the turning axis.

In one form, the trailing surface bounds part of the first circumferential gap.

In one form, the first arm has a radial length. The leading and trailing surfaces produce a wedge shape as viewed along the radial length of the first arm.

In one form, the trailing surface is defined by a plurality of flat surface portions.

In one form, the at least first and second arms make up at least part of a cutting head. The first shaping tool further includes an elongate driving body connected to the cutting head.

In one form, a reinforcing component connects between the first and second arms at locations on each of the first and second arms spaced radially from the turning axis.

In one form, at least one reinforcing component connects to each of the circumferentially adjacent of the at least first and second arms at locations on each of the circumferentially adjacent of the at least first and second arms spaced radially from the turning axis.

In one form, the at least first and second arms each has a radial distal end. The first turning tool further includes an arcuately-shaped reinforcing component that connects to each of the at least first and second arms at locations on each of the at least first and second arms spaced radially from the turning axis.

In one form, at least a majority of the length of the at least first and second cutting edges extends distally beyond the arcuately-shaped reinforcing component.

In one form, the arcuately-shaped component has axially spaced proximal and distal ends at an outer circumference. The at least first and second cutting edges each extends radially at least up to the outer circumference of the arcuately-shaped component at the distal end of the arcuately-shaped component.

In one form, the locations on each of the at least first and second arms are at or adjacent the radial distal ends of the at least first and second arms.

In one form, the arcuately-shaped reinforcing component has a continuous ring shape.

In one form, the arcuately-shaped reinforcing component has axially spaced leading and trailing ends and radially inwardly and outwardly facing surfaces. The radially inwardly facing surface has a radial dimension progressively reduced between the leading and trailing ends of the arcuately-shaped reinforcing component.

In one form, a guide passage is formed in the driving body and cutting head. The method of shaping a surface of a bone further includes the steps of: directing an elongate guide component into the bone; and, with the elongate guide component in the bone and directed into the guide passage, guidingly advancing the first shaping tool around the turning axis.

In one form, the first circumferential gap has a radially opening “V” shape as a viewed along the turning axis.

In one form, the at least first and second arms each has a radial distal end. The first shaping tool has a first diameter at the radial distal ends of the at least first and second arms. A majority of a circumference of the first shaping tool at the first diameter, as viewed along the turning axis, is defined cooperatively by circumferential gaps, corresponding to the first circumferential gap, defined between adjacent of the at least first and second arms.

In one form, there is a circumferential gap, corresponding to the first circumferential gap, between each of the adjacent first, second, and third arms.

In one form, the method of shaping a surface of a bone further includes the steps of: obtaining a rotary driver device; connecting the rotary driver device to the first shaping tool; and operating the rotary driver device to advance the shaping tool around the turning axis.

In one form, the at least first and second cutting edges are configured so that the flat bone surface shape resides in a plane that is perpendicular to the turning axis.

In one form, the lengths of the cutting edges on the first and second arms are non-straight.

In one form, the at least first and second arms make up at least part of a first cutting head. The first cutting head additionally includes at least one cutting component. As an incident of causing the at least first and second cutting edges to remove bone material and produce the flat surface shape, the at least one cutting component is caused to produce one of: a) a discrete projection of the bone above the flat bone surface shape; and b) a discrete depression within the bone beneath the flat bone surface shape.

In one form, the method of shaping a surface of a bone further includes the step of obtaining a second shaping tool having a second turning axis and a second cutting head configured to be urged against a bone while turning the second shaping tool around the second turning axis to thereby produce one of: a) a discrete depression in bone in the event that the first shaping tool is configured to produce a discrete projection; and b) a discrete depression in bone in the event that the first shaping tool is configured to produce a discrete projection.

In one form, the method of shaping a surface of a bone further includes the steps of: using the first shaping tool to form one of a discrete projection and a discrete depression at a first location on a first bone part; using the second shaping tool to form the other of a discrete projection and a discrete depression at a second location on a second bone part; and relatively moving the first and second bone parts to cause the discrete projection to move into the discrete depression.

In one form, the method of shaping a surface of a bone further includes the step of fusing the first and second bone parts with the discrete projection in the discrete depression.

In one form, the discrete projection and discrete depression have complementary shapes configured to make a keyed connection with the discrete projection moved into the discrete depression.

In one form, the discrete projection has an axis and is tapered in diameter along the axis to facilitate guided movement of the discrete projection into the discrete depression.

In one form, the second cutting head is configured to remove bone material and produce a flat surface shape in bone as the second cutting head is urged against bone while turning the shaping tool around the second turning axis.

In one form, the method of shaping a surface of a bone further includes the steps of: using the first shaping tool to form the flat bone surface shape and one of a discrete projection and a discrete depression at a first location on a first bone part; using the second shaping tool to form the flat surface shape and the other of a discrete projection and a discrete depression at a second location on a second bone part; and relatively moving the first and second bone parts to: a) place the flat bone surface shape formed by the first shaping tool on the first bone part into apposed relationship with the flat surface shape formed by the second shaping tool on the second bone part; and b) cause the discrete projection to move into the discrete depression.

In one form, each of the first and second shaping tools has a guide passage extending along a respective turning axis. The method of shaping a surface of a bone further includes the steps of: directing an elongate guide component into each of the first and second bone parts; directing the elongate guide component directed into the first bone part into the guide passage on the first shaping tool; directing the elongate guide component directed into the second bone part into the guide passage on the second shaping tool; and guiding the first and second shaping tools along respective elongate guide components as the discrete projection is moved into the discrete depression.

### BRIEF DESCRIPTION OF THE DRAWINGS

FIG. 1 is a plan view of two bones at an interphalangeal joint;

FIG. 2 is a view as in FIG. 1 wherein adjacent bone surfaces in FIG. 1 have been reconfigured by being shaped to produce flat surfaces that are placed in apposed relationship that is maintained by fusion;

FIG. 3 is a perspective view of a conventional cutting/shaping tool connected to a rotary driver, shown in schematic form;

FIG. 4 is a view of the cutting tool taken from a different perspective than in FIG. 3;

FIG. 5 is an enlarged, fragmentary, side elevation view of the distal region of the cutting tool in FIGS. 3 and 4 and showing a cutting head thereon;

FIG. 6 is a schematic representation of an elongate guide component that is usable in conjunction with the cutting tool in FIGS. 3-5;

FIG. 7 is a schematic representation of a bone with an exposed region that can be shaped, according to the present invention;

FIG. 8 is a schematic representation of a shaping tool usable to perform the inventive method;

FIG. 9 is a schematic representation showing additional details of the shaping tool in FIG. 8;

FIG. 10 is a schematic representation showing the shaping tool in FIGS. 8 and 9 in relationship to an elongate component usable to guide movement of the shaping tool;

FIG. 11 is a schematic representation of arms on the shaping tool in FIGS. 8-10 with at least one reinforcing component connected between the arms;

FIG. 12 is a perspective view of one exemplary form of the inventive shaping tool, as shown schematically in FIGS. 8-11;

FIG. 13 is an enlarged, fragmentary view of the shaping tool in FIG. 12 and taken from a different perspective;

FIG. 14 is a view of the portion of the shaping tool, as in FIG. 13, and from a side elevation perspective;

FIG. 15 is an end elevation view of the shaping tool in FIGS. 12-14;

FIG. 16 is a view of the inventive shaping tool as in FIG. 12 and taken from a different perspective;

FIG. 17 is a view of the inventive shaping tool, as in FIGS. 12 and 16, and taken from a different perspective;

FIG. 18 is a perspective view of a modified form of shaping tool, according to the present invention;

FIG. 19 is a side elevation view of the shaping tool in FIG. 18;

FIG. 20 is a side elevation view of a further modified form of shaping tool, according to the invention;

FIG. 21 is a flow diagram representation of a method of shaping a surface of a bone according to the invention;

FIG. 22 is a flow diagram representation of additional steps that might optionally be carried out in performing the inventive method;

FIG. 23 is a schematic representation of two shaping tools, according to the invention, modified from the shaping tool shown in FIGS. 13-19, and usable to form additional cooperating contours on cooperating bone parts;

FIG. 24 is a schematic depiction of different cooperating contours that are producible on bone using the shaping tools in FIG. 23;

FIG. 25 is a fragmentary elevation view of an exemplary form of the first shaping tool as shown in FIG. 23;

FIG. 26 is an inverted view as in FIG. 25 and showing an exemplary form of the second shaping tool shown schematically in FIG. 23;

FIG. 27 is a fragmentary and sectional view showing two different bone parts with contours produced using the shaping tools in FIGS. 25 and 26; and

FIG. 28 is a flow diagram representation of a method of shaping surfaces of bones, according to the present invention.

### DETAILED DESCRIPTION OF THE PREFERRED EMBODIMENT

The invention is directed to a method of shaping a surface 44 of a bone 46, as shown schematically in FIG. 7, on which a flat surface shape is to be either formed or modified.

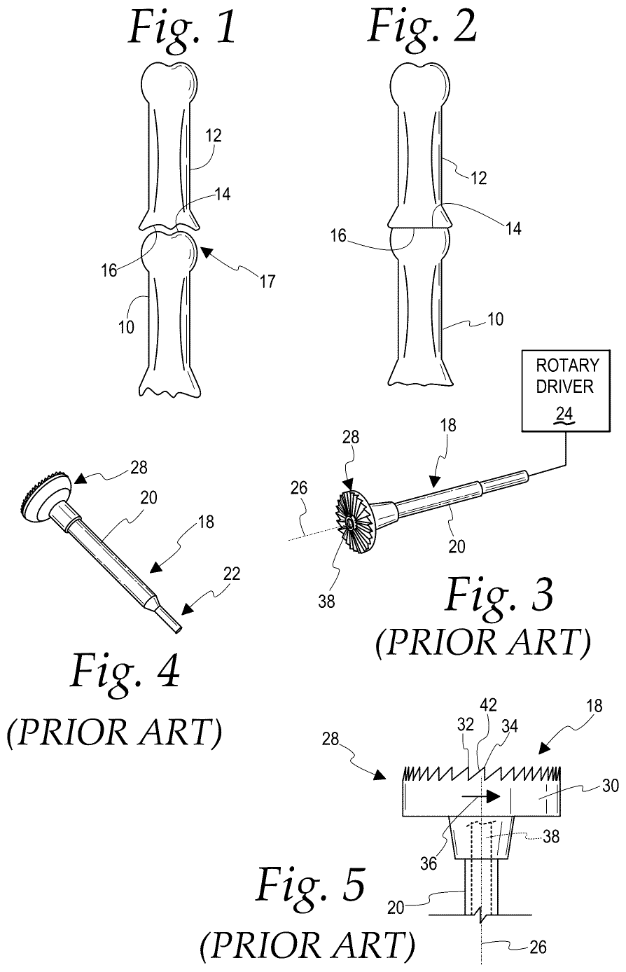

The schematic showing in FIG. 7 is intended to encompass virtually any bone 46 that is commonly reconfigured to perform different procedures; the exact forms of which are not critical to understanding the present invention. The procedure described with respect to FIGS. 1 and 2 above is representative in nature and should not be viewed to be in any way limiting. The inventive method can be practiced on any bone, at any location on a bone, and to perform any procedure conventionally requiring the formation, or reshaping, of a flat bone surface shape.

As shown schematically in FIG. 8, the invention contemplates that the method be carried out using a shaping/cutting tool 48 having a turning axis and axially spaced proximal and distal ends.

The shaping tool 48 has a plurality of discrete arms 50, with the shaping tool 48 depicted having at least representative first and second discrete arms 50a, 50b projecting away from the turning axis. A circumferential gap 52 is defined between circumferentially adjacent of the arms 50. As depicted, the gap 52 is shown between the first and second arms 50a, 50b, which is not a requirement.

The at least first circumferential gap 52 extends axially towards the proximal end of the shaping tool 48 to beyond the circumferentially adjacent of the at least first and second arms 50a, 50b.

The at least first and second arms 50 each has a cutting edge 54. As depicted, the first arm 50a has a cutting edge 54a, with the second arm 50b having a cutting edge 54b.

The cutting edges 54 move in a cutting path as the shaping tool 48 is advanced around the turning axis.

With the shaping tool 48 as depicted in FIG. 8, the at least first and second cutting edges 54 can be borne against the surface 44 of the bone 46 while advancing the shaping tool 48 around its axis, thereby causing: a) the cutting edges 54 to remove bone material/particles and produce a flat bone surface shape; and b) removed bone material/ particles to move axially away from the flat bone surface shape and into the circumferential gap 52.

As referenced, the “at least” first and second discrete arms can be any number of arms-two or greater. The depicted gap(s) 52 in FIG. 8 is not limited to being between the identified first and second arms 50a, 50b and a gap 52 may be between any and potentially all of the adjacent arms 50.

The gap 52 depicted can vary substantially in form and is in communication with a volume formed axially towards the proximal end of the shaping tool beyond the discrete arms 50 to accommodate at least some amount of generated bone particles so as to avoid buildup of particles between adjacent arms 50 where the gap 52 is located.

In one preferred form, as shown in FIG. 9, the arms 50 are part of a cutting head 56 with the gap(s) 52 extending fully through the cutting head 56 so as to have an axially unobstructed path into, through, and from the gap 52.

In one form, as also shown in FIG. 9, the shaping tool 48 is made up of the cutting head 56 and a driving body 58 which is fixed thereto can be engaged and turned by a rotary driver, as shown at 24 in FIG. 3.

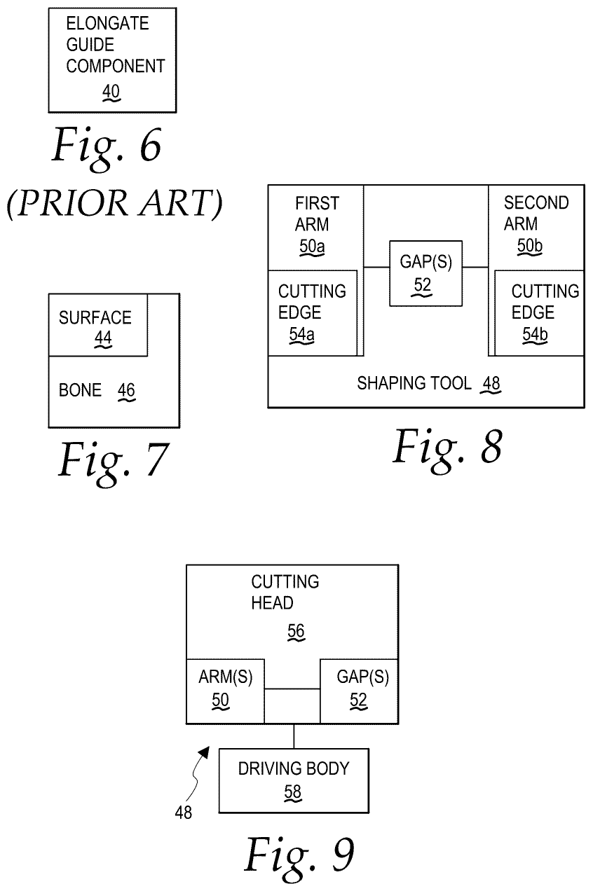

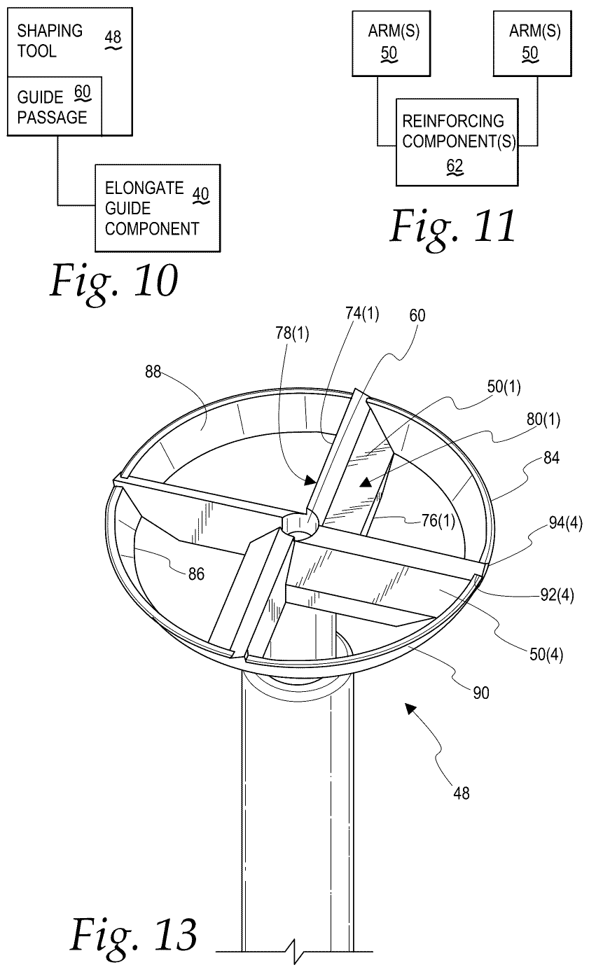

In one form, as shown in FIG. 10, the shaping tool 48 has a guide passage 60 extending axially at least partially therethrough to cooperate with the elongate guide component 40, as shown also in FIG. 6, in the same manner that the elongate guide component 40 in FIG. 6 is used with the tool 18 in FIGS. 3-5.

As shown in FIG. 11, at least one reinforcing component 62 connects between at least two adjacent arms 50. The reinforcing component 62 may take any shape—including without limitation, straight, curved, etc. While characterized as a “reinforcing component”, the reinforcing function may be effectively minimal, with the reinforcing component 62 performing other functions, as described below.

In one exemplary form, the reinforcing component 62 is arcuate and has a radius centered on the operating axis for the shaping tool 48. The arcuate shape may extend through less than 360° or may have a full ring shape.

The arcuately-shaped component 62 may be at any radial location on the arms 50, and in one preferred form does not extend beyond the radial dimension of the arms 50—although such a design is permissible.

The schematic depictions of components and their interaction are intended to encompass virtually an unlimited number of variations of the components, their interactions, and their use, with the exemplary forms and uses described hereinbelow being exemplary in nature only. Specific exemplary forms will now be described according to the invention.

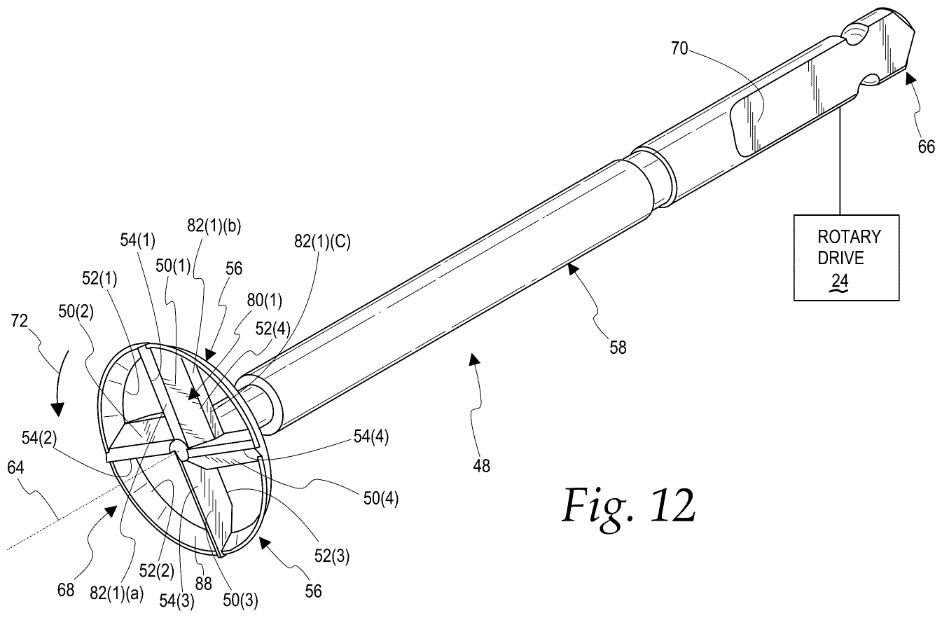

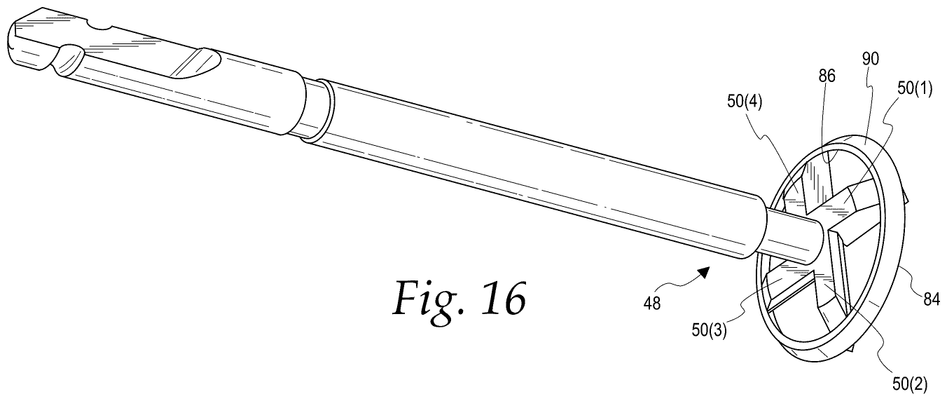

In FIGS. 12-17, one exemplary form of the shaping tool 48 is depicted. The shaping tool 48 has a cutting head 56 with an elongate driving body 58.

The shaping tool 48 has a lengthwise turning axis 64 and axially spaced proximal and distal ends 66, 68, respectively.

In this embodiment, the shaping tool 48 has four discrete arms 50(1), 50(2), 50(3), 50(4) which project radially away from the turning axis 64. The four arm design creates a symmetric shape with even weight distribution for smooth operation.

In this embodiment, there are like gaps 52 between all adjacent arms 50, identified as 52(1) between the arms 50(1) and 50(2); 52(2) between the arms 50(2) and 50(3); 52(3) between the arms 50(3) and 50(4); and 52(4) between the arms 50(4) and 50(1). The same gap configuration depicted is not required.

Each of the circumferential gaps 52 extends axially towards the proximal end 66 of the shaping tool 48 to beyond each of the discrete arms 50 into an open volume, as depicted, or potentially a confined volume.

On the shaping tool 48, the distal end 68 is the leading end of the shaping tool 48 at which cutting edges 54(1), 54(2), 54(3), 54(4) are defined respectively on the arms 50(1), 50(2), 50(3), 50(4).

With the gap construction depicted, bone material/particles removed by the cutting edges 54 are allowed to pass into each of the gaps 52 and move axially in an unobstructed path fully through and past the cutting head 56.

A fitting 70 is formed at the proximal end 66 of the driving body 58 and is releasably connectable to a rotary driver 24 which is operable to advance the shaping tool 48 in a driving direction, as indicated by the arrow 72, around the turning axis 64. As this occurs, each of the cutting edges 54 moves in a cutting path.

The arms 50 are configured so that by bearing the cutting edges 54 against the bone 46 while advancing the shaping tool 48 around the axis 64: a) the cutting edges 54 remove bone material and produce a flat bone surface shape; and b) cause removed bone material to move axially away from the flat bone surface shape and into the gaps 52.

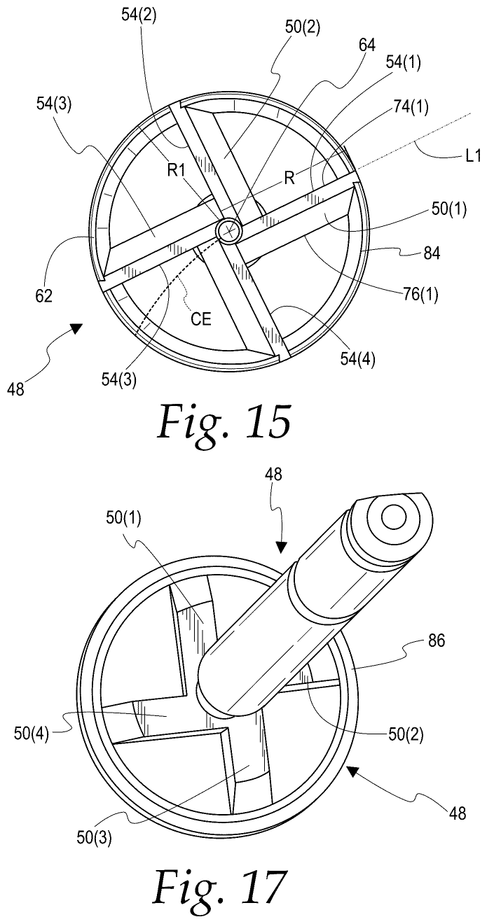

In this embodiment, all of the arms 50 and cutting edges 54 have the same configuration. For exemplary arm 50(1), the cutting edge 54(1) is substantially straight, extends in a line L1 (FIG. 15) that extends near or through the turning axis 64, and resides in a plane that is substantially orthogonal to the turning axis 64.

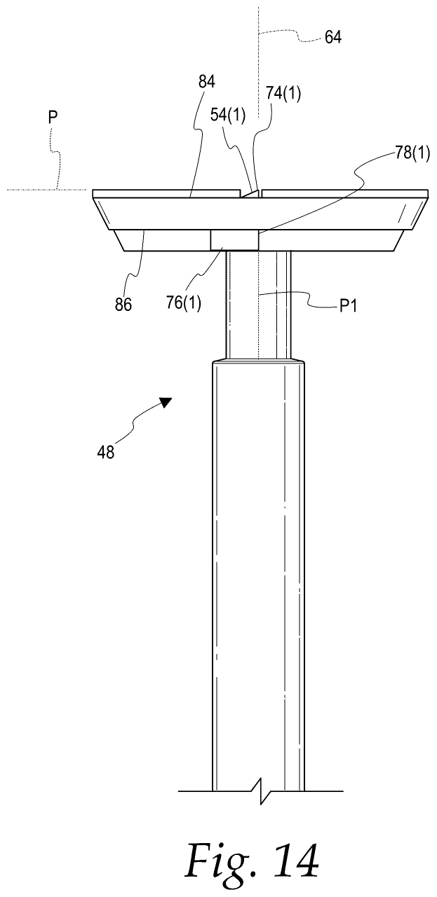

With this configuration, the cutting edges 54 all reside substantially within a single plane P (FIG. 14).

It is possible to construct the arms 50 and cutting edges 54 so that they have many different shapes which cooperatively produce the flat bone surface shape when the shaping tool 48 is operated.

As but one example, it is not required that the cutting edges 54 extend in straight lines. As seen in FIG. 15, the exemplary cutting edge 54(3) may have a non-straight shape, such as, without limitation, the curved shape indicated in dotted lines at CE.

Each of the arms 50 has an axial leading end 74 and a trailing end 76. The exemplary arm 50(1), as seen in FIG. 15, has a leading end 74(1) and a trailing end 76(1).

Each of the arms 50 has a radially projecting dimension, measured from the turning axis 64, identified for the representative first arm 50 in FIG. 15 at R. The radially projecting dimension of the first arm 50(1) is reduced between the leading end 74(1) and trailing end 76(1) as seen clearly from the FIG. 15 end perspective.

Each arm 50, and as seen for the representative arm 50(1) in FIG. 13, has circumferentially oppositely facing leading and trailing surfaces 78(1), 80(1), respectively, as seen in FIG. 13. The radially projecting dimension of the arm 50(1) between the oppositely facing leading and trailing surfaces 78(1), 80(1) changes between the leading end 74(1) and the trailing end 76(1)—being smaller at the trailing end 76(1).

The leading surface 78(1) of the arm 50(1) is substantially flat. In the depicted form, the leading surface 78(1) resides in a plane that is substantially parallel to the turning axis 64. In this form, while not required, the plane P1 of the leading surface 78(1) is substantially coincident with the turning axis 64.

The trailing surface 80(1) is defined by a plurality of flat surface portions 82(1)(a), 82(1)(b), 82(1)(c), as seen clearly in FIG. 12, with the surface portion 82(1)(c) being substantially parallel to the leading surface 78(1).

With this configuration, the first arm 50(1) has a wedge shape produced by the leading and trailing surfaces 78(1) and 80(1), respectively, as viewed along the radial length of the arm 50(1).

In this embodiment, each of the gaps 52 is bounded by a leading surface 78 of one arm 50 and a trailing surface 80 of a circumferentially adjacent arm 50.

Each of the circumferential gaps 52 has a radially opening “V” shape as viewed along the turning axis 64.

The reinforcing component(s) 62, shown schematically in FIG. 11, may take virtually an unlimited number of different forms and shapes.

In this embodiment, the reinforcing component 62 is in the form of a continuous ring that engages each of the arms 50 at a location spaced equidistantly radially from the turning axis 64. As noted, the reinforcing component 62 does not have to have this ring shape or connect between all of the arms 50.

As one example, the continuous ring shape may be interrupted so that there are arcuately-shaped reinforcing components between a pair of arms at diametrically opposite locations.

The component 62 may be made in pieces or as a single piece, as depicted. The component 62 has an axial leading end 84, a trailing end 86, a radially inwardly facing surface 88, and a radially outwardly facing surface 90. The surface 88 is concave, with the surface 90 convex. The surface 88 reduces in radial dimension between the leading end 84 and trailing end 86 to at least nominally match the shapes of the radial distal ends of the arms 50.

The cutting edges 54 extend distally beyond the leading end 84 of the component 62. The amount of extension of the cutting edges 54 past the leading end 84 determines initial depth of cutting. In the event that the cutting edges 54 do not reside fully in the same plane, it is desirable that at least a majority of the lengths of the cutting edges 54 extend distally beyond the leading end 84 of the component 62. It is also preferred that the cutting edges 54 extend up to, or distally beyond, the component 62 at the radial distal ends of the arms 50 so that the component 62 does not interfere with bone cutting thereat.

As seen for the exemplary arm 50(4) in FIG. 13, there is a notch 92(4) in which the leading end 84 of the component 62 nests to allow the free end 94(4) to extend beyond, or be substantially flush, with the outer circumference of the surface 90 on the component 62.

The continuous ring shape of the component 62 provides a number of advantages. It reinforces all of the arms 50, maintains their relationship, and avoids unwanted flexing thereof, even when relatively long arms 50 are desired to have a relatively large diameter cutting/shaping footprint.

The ring shape also gives the surgeon a clear visual indication of the cutting/shaping footprint.

The ring shape also shields adjacent bones and tissue against inadvertent contact with the arms 50 that might cause damage thereto.

With the four arm arrangement, when viewing the tool from the FIG. 15 perspective, a majority of the circumference of the cutting tool at a distance R1 from the turning axis 64 to the free arm ends 94 is defined cooperatively by the gaps 52, which facilitate pass-through of bone particles and avoid any buildup that might interfere with the performance of the cutting edges 54.

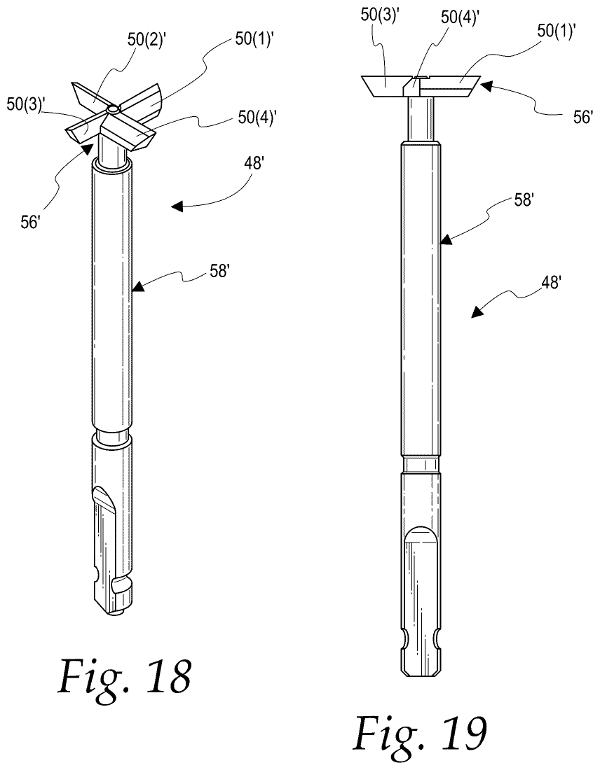

A modified form of the shaping tool is shown at 48′ in FIGS. 18 and 19. The shaping tool 48′ has a cutting head 56′ and driving body 58′ that are functionally the same as the driving body 58 and cutting head 56 on the shaping tool 48.

Further, the arms 50(1)′, 50(2)′, 50(3)′, and 50(4)′ may have the same shape as the arms 50 on the shaping tool 48.

The only significant difference between the shaping tool 48′ and the shaping tool 48 is that the reinforcing component 62 has been eliminated.

With the reinforcing component 62 absent, the previously described wedge shape of the exemplary arm 50(4)′, as viewed along the length of that arm, can be more clearly seen.

A further modified form of shaping tool, according to the present invention, is shown at 48″ in FIG. 20 with four arms 50″. Three arms 50(1)″, 50(3)″, and 50(4)″ are visible in FIG. 20.

The primary difference between the shaping tool 48″ and the shaping tool 48′ is that the arms 50″ have a shorter radially projecting dimension than the arms 50′. Accordingly, the shaping tool 48″ cuts with a smaller diameter footprint than the shaping tools 48, 48′.

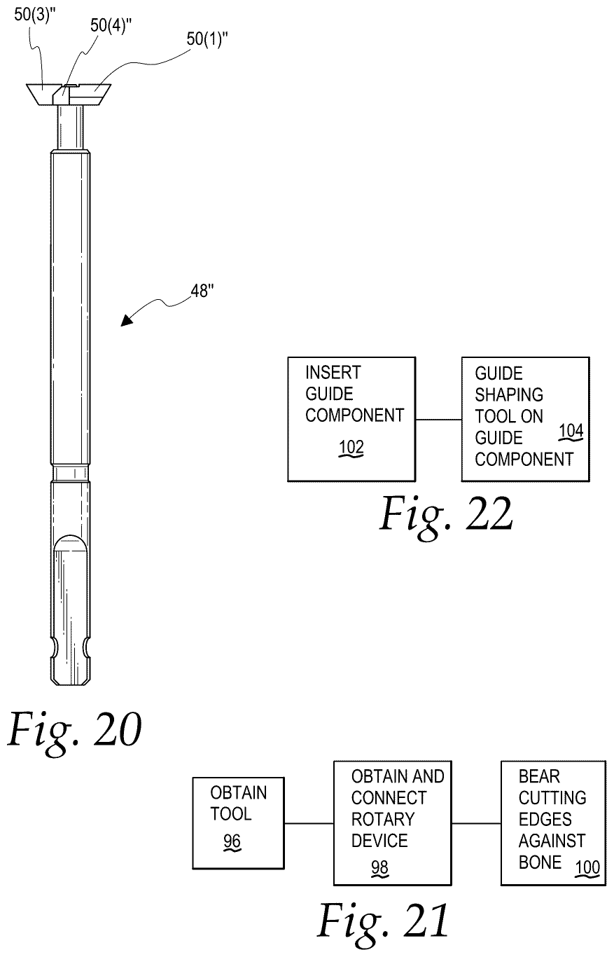

With the shaping tool 48, a method of shaping a surface of a bone, according to the invention, can be carried out as shown in flow diagram form in FIG. 21.

As shown at block 96, a shaping tool, as described above, is obtained.

As shown at block 98, a rotary driver device, as shown in FIG. 12, is obtained and connected to the shaping tool.

As shown at block 100, the cutting edges on the shaping tool are borne against bone while the rotary driver device is operated to cause: a) the cutting edges on the discrete arms to remove bone material and produce a flat bone surface shape; and b) removed bone material to move axially away from the bone surface and through the circumferential gaps on the shaping tool so as to avoid accumulation on the shaping tool and particularly at the cutting head.

As previously mentioned, the shaping tool 48 may be operated freehand or, alternatively, as shown in FIG. 22 at block 102, a guide component as shown in FIG. 6 may be inserted into a bone, after which, as shown at block 104, the shaping tool may be engaged with the guiding component and guidingly moved therearound to control both location and orientation of the flat bone surface shape produced.

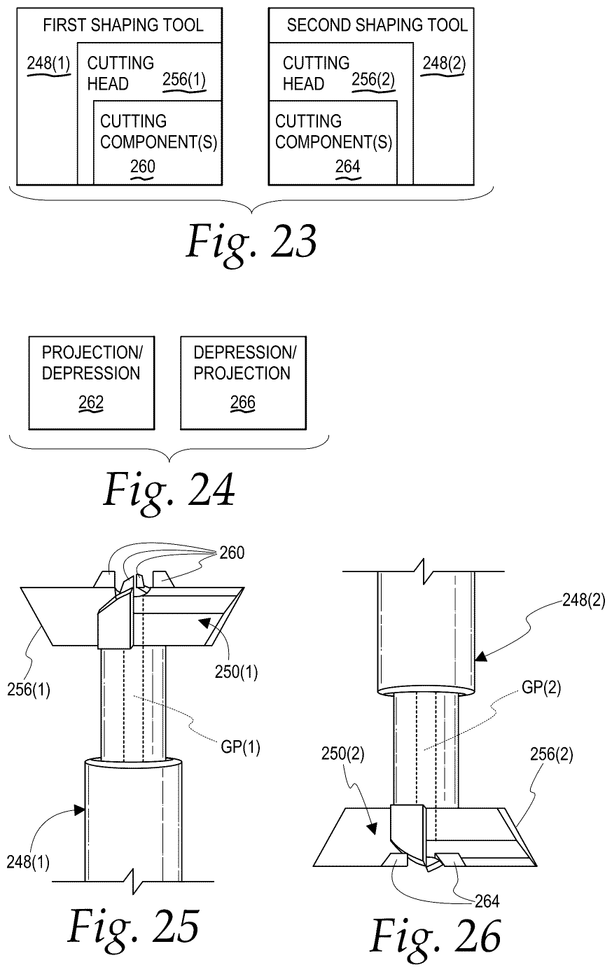

One variation of the invention is shown schematically in FIGS. 23 and 24.

A first shaping tool 248(1), corresponding to the previously described shaping tool 48, has a cutting head 256(1), corresponding to the aforementioned cutting head 256, with the cutting head 256(1) configured to be operable in substantially the same manner as the cutting head 56 to produce a contour in bone in addition to the flat bone surface shape. The first shaping tool 248(1) can be operated in substantially the same manner as the shaping tool 48.

By incorporating at least one additional cutting component 260, as the first shaping tool 248(1) is operated, the additional contour produced is one of a discrete projection/depression, as shown schematically at 262 in FIG. 24.

The first shaping tool 248(1) can be used in conjunction with a second shaping tool 248(2) with a cutting head 256(2) that incorporates at least one additional cutting component 264. The second shaping tool 248(2) can have, but is not required to have, the same basic construction as the first shaping tool 248(1), with the exception that the cutting component(s) 264 incorporated into the cutting head 256(2) produces a discrete depression/projection in bone, as identified schematically at 266 in FIG. 24.

The schematic depiction of the projection/depression 262 and depression/projection 266 is intended to encompass virtually any complementary male/female shapes that can be produced through a rotary shaping tool.

It is also possible that multiple projections/depressions 262 and depressions/projections 266 can be provided in each of the cooperating bone parts in which they are formed.

What is desirable is that the projection/depression 262 and depression/projection 266 interact as bone locations on separate bone parts, at which the projection/depression 262 and depression/projection 266 are formed, that are brought into adjacent relationship, or apposed relationship, so as to thereby confine at least one dimension of relative movement between those bone parts once they are brought together, as in anticipation of fusion.

One exemplary form for the cutting heads 256(1) and 256(2) will be described with respect to FIGS. 25-27, with it being understood that these are not in any way limiting but intended to be exemplary in nature only.

For purposes of simplicity, the first shaping tool 248(1) will be identified as having the cutting head 256(1) with cutting components 260 that produce a first depression 262 in a first bone part 268.

The second shaping tool 248(2) has a cutting head 256(2) with cutting components 264 configured to produce a discrete projection 266 in a second bone part 272.

The first shaping tool 248(1) is shown with arms 250(1) with the same basic shape as the arms 50 (previously described). Likewise, the second shaping tool 248(2) may have arms 250(2) corresponding to the arms 50, in terms of shape and function.

However, the second shaping tool 248(2) may not be required to form a flat bone surface shape in addition to the projection 266.

The details of the cutting components 260, 264 could be clearly arrived at by one skilled in the art to produce desired cooperating shapes for the depression 262 and projection 266. Thus, no such details will be described herein. As depicted, the cutting components 260 and the cutting components 264 each consists of a series of edges which are aligned and cooperate to produce the depicted bone shapes.

In the exemplary form, the projection 266 tapers away from a flat surface shape 274. The depression 262 is formed in the bone part 268 beneath the flat bone surface shape 276 defined by the first shaping tool 248(1).

The tapering surface 278 of the projection 266 is guided into the depression 262 by a complementary shape of a bounding surface 280.

In one form, locations on the bone parts 268, 272, at which the depression 262 and projection 266 are located, are moved towards each other to place the bone surface shapes 274, 276 into apposed relationship simultaneously as the surface 278 seats adjacent to, or against, the surface 280. With this arrangement, the projection 266 within the depression 262 confines at least relative translational movement between the bone parts 268, 272 generally parallel to the planes of the surface shapes 274, 276.

It should be noted that the surface shapes 274, 276 and surfaces 278, 280 are not required to simultaneously abut but may be placed in adjacent relationship to achieve alignment objectives and avoid excessive shifting of the bone parts 268, 272 relative to each other.

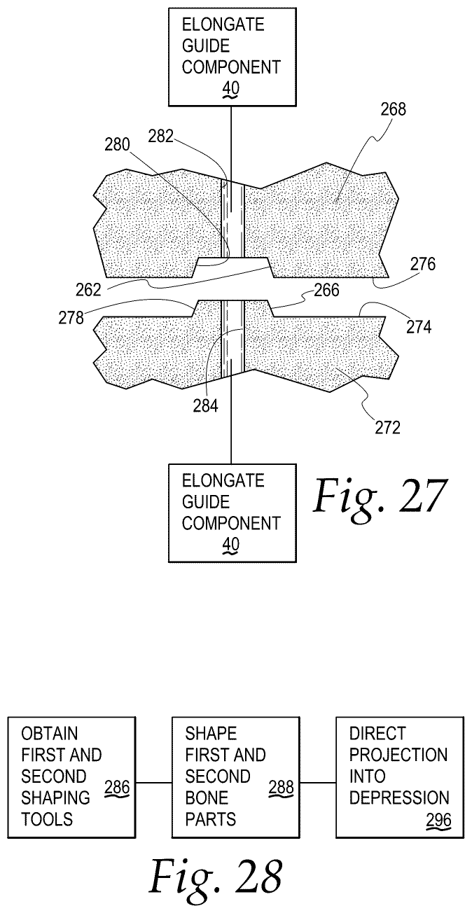

As shown in FIG. 27, each of the bone parts 268, 272 has a bore 282, 284, respectively, to receive separate, elongate guide components 40, as previously described.

The shaping tool 248(1) has a guide passage GP(1), corresponding to the guide passage 60, previously described, with the shaping tool 248(2) having a guide passage GP(2) likewise corresponding to the guide passage 60.

By using elongate guide components 40 on each of the bone parts 268, 272, the respective shaping tools 248(1), 248(2) can be guided therealong to precisely locate the depression 262 and projection 266 and ensure their proper alignment when the bone parts 268, 272 are relatively moved to advance the projection 266 into the depression 262.

The tapered arrangement of the surfaces 278, 280 consistently guides the bone parts 268, 272 in a desired end relationship, as preparatory to fusion.

With the first and second shaping tools 248(1), 248(2), the aforementioned method can be carried out in substantially the same manner as described above, with the exception that rather than using a single shaping tool to reconfigure separate bone parts, the different cutting tools 248(1), 248(2) are used, one each on the separate bone parts.

Accordingly, the method can be carried out as shown in flow diagram form in FIG. 28.

As shown at block 286, the first and second shaping tools are obtained.

As shown at block 288, one of the shaping tools is used to reconfigure one of two cooperating bone parts with the other of the shaping tools used to reconfigure the other of the cooperating bone parts. This configuration produces at least one projection on one of the bone parts and a complementary depression on the other of the bone parts.

As shown at block 290, the bone parts are relatively moved, thereby causing the projection(s) to be directed into the depression(s).

The cooperating projection 266 and depression 262, in addition to preventing shifting of the bone parts in translation transversely to the lengths, may, in conjunction with the cooperating apposing surface portions, to some extent limit angulation between the bone parts 268, 272.

The foregoing disclosure of specific embodiments is intended to be illustrative of the broad concepts comprehended by the invention.

1. A method of shaping a surface of a bone, the method comprising the steps of:
obtaining a first shaping tool having a turning axis, and axially spaced proximal and distal ends, the first shaping tool comprising:
at least first and second discrete arms projecting away from the turning axis with at least a first circumferential gap defined between circumferentially adjacent of the at least first and second arms,
the at least first circumferential gap extending axially towards the proximal end of the first shaping tool to beyond the circumferentially adjacent of the at least first and second arms,
the at least first and second arms each having a cutting edge,
the cutting edge on each of the at least first and second arms moving in a cutting path as the first shaping tool is advanced around the turning axis; and
bearing the at least first and second cutting edges against the bone while advancing the first shaping tool around the turning axis and thereby causing: a) the at least first and second cutting edges to remove bone material and produce a flat bone surface shape; and b) removed bone material to move axially away from the flat bone surface shape and into the first circumferential gap.

2. The method of shaping a surface of a bone according to claim 1, wherein the cutting edges on the first and second arms have lengths projecting away from the turning axis that reside substantially in a single plane.

3. The method of shaping a surface of a bone according to claim 1, wherein the at least first and second arms comprise at least a third arm.

4. The method of shaping a surface of a bone according to claim 1, wherein the at least first and second arms comprise at least a third arm and a fourth arm.

5. The method of shaping a surface of a bone according to claim 2, wherein the lengths of the cutting edges on the first and second arms are substantially straight and respectively extend along first and second lines.

6. The method of shaping a surface of a bone according to claim 5, wherein the first and second lines intersect the turning axis for the first shaping tool.

7. The method of shaping a surface of a bone according to claim 1, wherein the at least first and second arms are part of a cutting head and the first circumferential gap is axially unobstructed whereby removed bone material moves axially away from the flat bone surface shape and into and fully through the first circumferential gap and fully through the cutting head.

8. The method of shaping a surface of a bone according to claim 1, wherein the first circumferential gap is defined between the first and second arms, the distal end of the first shaping tool is a leading end, the first arm has a leading end and a trailing end, the first arm has a radial distal end and a radially projecting dimension of the first arm to the radial distal end of the first arm is reduced between the leading end of the first arm and the trailing end of the first arm.

9. The method of shaping a surface of a bone according to claim 8, wherein the first arm has circumferentially oppositely facing leading and trailing surfaces and the radially projecting dimension of the first arm between the oppositely facing leading and trailing surfaces changes between the leading end and trailing end of the first arm.

10. The method of shaping a surface of a bone according to claim 9, wherein the leading surface of the first arm is substantially flat.

11. The method of shaping a surface of a bone according to claim 10, wherein the leading surface of the first arm resides in a plane that is substantially parallel to the turning axis.

12. The method of shaping a surface of a bone according to claim 11, wherein the plane is substantially coincident with the turning axis.

13. The method of shaping a surface of a bone according to claim 9, wherein the trailing surface bounds part of the first circumferential gap.

14. The method of shaping a surface of a bone according to claim 13, wherein the first arm has a radial length and the leading and trailing surfaces produce a wedge shape as viewed along the radial length of the first arm.

15. The method of shaping a surface of a bone according to claim 9, wherein the trailing surface is defined by a plurality of flat surface portions.

16. The method of shaping a surface of a bone according to claim 1, wherein the at least first and second arms make up at least part of a cutting head and the first shaping tool further comprises an elongate driving body connected to the cutting head.

17. The method of shaping a surface of a bone according to claim 1 wherein a reinforcing component connects between the first and second arms at locations on each of the first and second arms spaced radially from the turning axis.

18. The method of shaping a surface of a bone according to claim 1 wherein at least one reinforcing component connects to each of the circumferentially adjacent of the at least first and second arms at locations on each of the circumferentially adjacent of the at least first and second arms spaced radially from the turning axis.

19. The method of shaping a surface of a bone according to claim 1 wherein the at least first and second arms each has a radial distal end and the first turning tool further comprises an arcuately-shaped reinforcing component that connects to each of the at least first and second arms at locations on each of the at least first and second arms spaced radially from the turning axis.

20. The method of shaping a surface of a bone according to claim 19 wherein at least a majority of the length of the at least first and second cutting edges extends distally beyond the arcuately-shaped reinforcing component.

21. The method of shaping a surface of a bone according to claim 20 wherein the arcuately-shaped component has axially spaced proximal and distal ends at an outer circumference, and the at least first and second cutting edges each extends radially at least up to the outer circumference of the arcuately-shaped component at the distal end of the arcuately-shaped component.

22. The method of shaping a surface of a bone according to claim 21 wherein the locations on each of the at least first and second arms are at or adjacent the radial distal ends of the at least first and second arms.

23. The method of shaping a surface of a bone according to claim 22 wherein the arcuately-shaped reinforcing component has a continuous ring shape.

24. The method of shaping a surface of a bone according to claim 23 wherein the arcuately-shaped reinforcing component has axially spaced leading and trailing ends and radially inwardly and outwardly facing surfaces, the radially inwardly facing surface has a radial dimension progressively reduced between the leading and trailing ends of the arcuately-shaped reinforcing component.

25. The method of shaping a surface of a bone according to claim 16, wherein a guide passage is formed in the driving body and cutting head and further comprising the steps of directing an elongate guide component into the bone, and with the elongate guide component in the bone and directed into the guide passage, guidingly advancing the first shaping tool around the turning axis.

26. The method of shaping a surface of a bone of claim 1, wherein the first circumferential gap has a radially opening “V” shape as a viewed along the turning axis.

27. The method of shaping a surface of a bone according to claim 1, wherein the at least first and second arms each has a radial distal end, the first shaping tool has a first diameter at the radial distal ends of the at least first and second arms, and a majority of a circumference of the first shaping tool at the first diameter, as viewed along the turning axis, is defined cooperatively by circumferential gaps, corresponding to the first circumferential gap, defined between adjacent of the at least first and second arms.

28. The method of shaping a surface of a bone according to claim 4, wherein there is a circumferential gap, corresponding to the first circumferential gap, between each of the adjacent first, second, and third arms.

29. The method of shaping a surface of a bone according to claim 1, further comprising the steps of obtaining a rotary driver device, connecting the rotary driver device to the first shaping tool, and operating the rotary driver device to advance the first shaping tool around the turning axis.

30. The method of shaping a surface of a bone according to claim 1 wherein the at least first and second cutting edges are configured so that the flat bone surface shape resides in a plane that is perpendicular to the turning axis.

31. The method of shaping a surface of a bone according to claim 2 wherein the lengths of the cutting edges on the first and second arms are non-straight.

32. The method of shaping a surface of a bone according to claim 1 wherein the at least first and second arms make up at least part of a first cutting head and the first cutting head additionally comprises at least one cutting component, and as an incident of causing the at least first and second cutting edges to remove bone material and produce the flat surface shape, the at least one cutting component is caused to produce one of: a) a discrete projection of the bone above the flat bone surface shape; and b) a discrete depression within the bone beneath the flat bone surface shape.

33. The method of shaping a surface of a bone according to claim 32 further comprising the step of obtaining a second shaping tool having a second turning axis and a second cutting head configured to be urged against a bone while turning the second shaping tool around the second turning axis to thereby produce one of: a) a discrete depression in bone in the event that the first shaping tool is configured to produce a discrete projection; and b) a discrete depression in bone in the event that the first shaping tool is configured to produce a discrete projection.

34. The method of shaping a surface of a bone according to claim 33 further comprising the steps of using the first shaping tool to form one of a discrete projection and a discrete depression at a first location on a first bone part, using the second shaping tool to form the other of a discrete projection and a discrete depression at a second location on a second bone part, and relatively moving the first and second bone parts to cause the discrete projection to move into the discrete depression.

35. The method of shaping a surface of a bone according to claim 34 further comprising the step of fusing the first and second bone parts with the discrete projection in the discrete depression.

36. The method of shaping a surface of a bone according to claim 34 wherein the discrete projection and discrete depression have complementary shapes configured to make a keyed connection with the discrete projection moved into the discrete depression.

37. The method of shaping a surface of a bone according to claim 34 wherein the discrete projection has an axis and is tapered in diameter along the axis to facilitate guided movement of the discrete projection into the discrete depression.

38. The method of shaping a surface of a bone according to claim 33 wherein the second cutting head is configured to remove bone material and produce a flat surface shape in bone as the second cutting head is urged against bone while turning the shaping tool around the second turning axis.

39. The method of shaping a surface of a bone according to claim 38 further comprising the steps of using the first shaping tool to form the flat bone surface shape and one of a discrete projection and a discrete depression at a first location on a first bone part, using the second shaping tool to form the flat surface shape and the other of a discrete projection and a discrete depression at a second location on a second bone part, and relatively moving the first and second bone parts to: a) place the flat bone surface shape formed by the first shaping tool on the first bone part into apposed relationship with the flat surface shape formed by the second shaping tool on the second bone part; and b) cause the discrete projection to move into the discrete depression.

40. The method of shaping a surface of a bone according to claim 34 wherein each of the first and second shaping tools has a guide passage extending along a respective turning axis and further comprising the steps of directing an elongate guide component into each of the first and second bone parts, directing the elongate guide component directed into the first bone part into the guide passage on the first shaping tool, directing the elongate guide component directed into the second bone part into the guide passage on the second shaping tool, and guiding the first and second shaping tools along respective elongate guide components as the discrete projection is moved into the discrete depression.
